(19) United States
(12) Patent Application Publication
(10) Pub. No.: US 2004/0030377 A1
(43) Pub. Date: Feb. 12, 2004

(54) Medicated polymer-coated stent assembly

(76) Inventors: Alexander Dubson, Hadera (IL); Eli Bar, Moshav Megadim (IL)

Correspondence Address: G E Ehrlich, Anthony Castorina, Suite 207, 2001 Jefferson Davis Highway, Arlington, VA, 22202, US

(21) Appl. No.: 10/433,620

(22) PCT Filed: Dec. 17, 2001
(86) PCT No.: PCT/IL01/01171

(30) Foreign Application Priority Data
Oct. 19, 2001 (US) 09982017

(51) Int. Cl. A61F 2/06

(52) U.S. Cl. 623/1.13; 264/638

(57) ABSTRACT

A stent assembly comprising an expensible tubular supporting element and at least one coat of electrospun polymer fibers, each of the at least one coat having a predetermined porosity, the at least one coat including at least one pharmaceutical agent incorporated therein for delivery of the at least one pharmaceutical agent into a body vasculature during or after implantation of the stent assembly within the body vasculature.

### FIELD AND BACKGROUND OF THE INVENTION

The present invention relates to an implantable stent, and, more particularly, to a medicated polymer-coated stent assembly, implantable within a blood vessel designed for delivering a pharmaceutical agent to the surrounding tissues.

Coronary heart disease may result in stenosis, which results in the narrowing or constriction of an artery. Percutaneous coronary intervention (PCI) including balloon angioplasty and stent deployment is currently a mainstay in the treatment of coronary heart disease. This treatment is often associated with acute complications such as late restenosis of angioplastied coronary lesions.

Restenosis refers to the reclosure of a previously stenosed and subsequently dilated peripheral or coronary blood vessel. Restenosis results from an acssesive natural healing process that takes place in response to arterial injuries inherent to angioplasty procedures. This natural healing process involves migration and proliferation of cells. In restenosis this natural healing process continues, sometimes until a complete reclusion of the vessel occurs.

A common solution to restonosis is intercoronary stenting, which is intended to provide the coronary with radial support and thereby prevent constriction. Nevertheless, clinical data indicates that stents are usually unable to prevent late restenosis beginning at about three months following an angioplasty procedure.

To date, attempts have been made to treat restenosis by systemic administration of drugs, and sometimes by intravascular irradiation of the angioplastied artery, however these attempts have not been successful. Hence, current research is being shifted gradually to the local administration of various pharmaceutical agents at the site of an arterial injury resulting from angioplasty. The advantages gained by local therapy include higher concentrations of the drug at the actual injury site. One example of such treatment is local drug delivery of toxic drugs such as taxol and rapamycin to the vessel site via a catheter-based delivery system. However, local treatment systems dispensing a medication on a one-shot basis cannot efficiently prevent late restenosis.

Numerous attempts to develop stents with a local drug-distribution function have been made, most of which are variances of the so called stent graft, a metal stent covered with polymer envelope, containing anti-coagulant and/or anti-proliferative medicaments. The therapeutic action of stent grafts is based on gradual decomposition of biodegradable polymers under the effect of aggressive biological medium and drug liberation into the tissues which is in direct contact with the stent graft location. Drug-loaded polymer can be applied by spraying or by dipping the stent graft into a solution or melt, as disclosed, for example, in U.S. Pat. Nos. 5,383,922, 5,824,048, 5,624,411 and 5,733,327. Additional method for providing a drug-loaded polymer is disclosed in U.S. Pat. Nos. 5,637,113 and 5,766,710, where a pre-fabricated film is attached to the stent. Other methods, such as deposition via photo polymerization, plasma polymerization and the like, are also known in the art and are described in, e.g., U.S. Pat. Nos. 3,525,745, 5,609,629 and 5,824,049.

Stent grafts with fiber polymer coating promote preparation of porous coatings with better grafting and highly developed surface. U.S. Pat. No. 5,549,663 discloses a stent graft having a coating made of polyurethane fibers which are applied using conventional wet spinning techniques. Prior to the covering process, a medication is introduced into the polymer.

A more promising method for stent coating is electrospinning. Electrospinning is a method for the manufacture of ultra-thin synthetic fibers which reduces the number of technological operations required in the manufacturing process and improves the product being manufactured in more than one way. The use of electrospinning for stent coating permits to obtain durable coating with wide range of fiber thickness (from tens of nanometers to tens of micrometers), achieves exceptional homogeneity, smoothness and desired porosity distribution along the coating thickness. Stents themselves do not encourage normal cellular invasion and therefore can lead to an undisciplined development of cells in the metal mesh of the stent, giving rise to cellular hyperplasia. When a stent is electrospinningly coated by a graft of a porous structure, the pores of the graft component are invaded by cellular tissues from the region of the artery surrounding the stent graft. Moreover, diversified polymers with various biochemical and physico-mechanical properties can be used in stent coating. Examples of electrospinning methods in stent graft manufacturing are found in U.S. Pat. Nos. 5,639,278, 5,723,004, 5,948,018, 5,632,772 and 5,855,598.

In is known that the electrospinning technique is rather sensitive to the changes in the electrophysical and Theological parameters of the solution being used in the coating process. In addition, incorporation of drugs into the polymer in a sufficient concentration, so as to achieve a therapeutic effect, reduces the efficiency of the electrospinning process. Still in addition, drug introduction into a polymer reduces the mechanical properties of the resulting coat. Although this drawback is somewhat negligible in relatively thick films, for submicron fibers made film this effect may be adverse.

Beside restenosis, PCI involves the risk of vessel damage during stent implantation. This risk may be better understood by considering the nature of the defect in the artery, which the stent is intended to resolve.

Arteriosclerosis or hardening of the arteries is a widespread disease involving practically all arteries of the body including the coronary arteries. Arteriosclerosis plaques adhere to the walls of the arteries and build up in the course of time to increasingly narrow and constrict the lumens of the arteries. An appropriate procedure to eradicate this constriction is balloon angioplasty, and/or stent placement. In the latter procedure, a stent is transported by a balloon catheter, known as a stent delivery device, to the defective site in the artery and then expanded radially by the balloon to dilate the site and thereby enlarge the passage through the artery.

As the balloon and/or stent expands, it then cracks the plaques on the wall of the artery and produces shards or fragments whose sharp edges cut into the tissue. This causes internal bleeding and a possible local infection, which if not adequately treated, may spread and adversely affect other parts of the body.

Local infections in the region of the defective site in an artery do not lend themselves to treatment by injecting an antibiotic into the blood stream of the patient, for such treatment is not usually effective against localized infections. A more common approach to this problem is to coat the wire mesh of the stent with a therapeutic agent which makes contact with the infected region. As stated, this is a one-shot treatment whereas to knock out infections, it may be necessary to administer an antibiotic and/or other therapeutic agents for several hours or days, or even months.

The risk of vessel damage during stent implantation may be lowered through the use of a soft stent serving to improve the biological interface between the stent and the artery and thereby reduce the risk that the stent will inflict damage during implantation. Examples of polymeric stents or stent coatings with biocompatible fibers are found in, for example, U.S. Pat. Nos. 6,001,125, 5,376,117 and 5,628,788, all of which are hereby incorporated by reference.

U.S. Pat. No. 5,948,018 discloses a graft composed of an expansible stent component covered by an elastomeric polymeric graft component which, because of its stretchability, does not inhibit expansion of the stent. The graft component is fabricated by electrospinning to achieve porosity hence to facilitate normal cellular growth. However, U.S. Pat. No. 5,948,018 fails to address injuries inflicted by the stent in the course of its implantation on the delicate tissues of the artery. These injuries may result in a local infection at the site of the implantation, or lead to other disorders which, unless treated effectively, can cancel out the benefits of the implant.

Additional prior art of interest include: Murphy et al. “Percutaneous Polymeric Stents in Porcine Coronary Arteries”, Circulation 86: 1596-1604, 1992; Jeong et al. “Does Heparin Release Coating of the Wallstent limit Thrombosis and Platelet Deposition?”, Circulation 92: 173A, 1995; and Wiedermann S. C. “Intercoronary Irradiation Markedly Reduces Necintimal Proliferation after Balloon Angioplasty in Swine” Amer. Col. Cardiol. 25: 1451-1456, 1995.

There is thus a widely recognized need for, and it would be highly advantageous to have, an efficient and reliable medicated polymer-coated stent assembly, which is implantable within a blood vessel and is designed for delivering a pharmaceutical agent to the surrounding tissues, which is devoid of the above limitations.

### SUMMARY OF THE INVENTION

According to one aspect of the present invention there is provided a stent assembly comprising an expansible tubular supporting element and at least one coat of electrospun polymer fibers, each of the at least one coat having a predetermined porosity, the at least one coat including at least one pharmaceutical agent incorporated therein for delivery of the at least one pharmaceutical agent into a body vasculature during or after implantation of the stent assembly within the body vasculature.

According to another aspect of the present invention there is provided a method of producing a stent assembly, the method comprising: (a) electrospinning a first liquefied polymer onto an expensible tubular supporting element, thereby coating the tubular supporting element with a first coat having a predetermined porosity; and (b) incorporating at least one pharmaceutical agent into the first coat.

According to yet another aspect of the present invention there is provided a method of treating a constricted blood vessel, the method comprising placing a stent assembly in the constricted blood vessel, the stent assembly comprises an expensible tubular supporting element and at least one coat of electrospun polymer fibers, each of the at least one coat having a predetermined porosity, the at least one coat including at least one pharmaceutical agent incorporated therein for delivery of the at least one pharmaceutical agent into a body vasculature during or after implantation of the stent assembly within the body vasculature.

According to still another aspect of the present invention there is provided a method of dilating a constricted blood vessel, the method comprising: (a) providing a stent assembly comprises an expansible tubular supporting element and at least one coat of electrospun polymer fibers, each of the at least one coat having a predetermined porosity, the at least one coat including at least one pharmaceutical agent incorporated therein; (b) placing the stent assembly to a constricted region in the constricted blood vessel; and (c) radially expanding the stent assembly within the blood vessel so as to dilate the constricted region and to allow blood flow through the blood vessel.

According to an additional aspect of the present invention there is provided a method of coating a medical implant, implantable in a body, the method comprising: (a) electrospinning a first liquefied polymer onto the medical implant, thereby coating the medical implant with a first coat having a predetermined porosity; and (b) incorporating at least one pharmaceutical agent into the first coat; thereby providing a coated medical implant.

According to further features in preferred embodiments of the invention described below, the at least one pharmaceutical agent is mixed with the liquefied polymer prior to the step of electrospinning, hence the step of incorporating the at least one pharmaceutical agent into the first coat is concomitant with the electrospinning.

According to still further features in the described preferred embodiments the medical implant is selected from the group consisting of a graft, a patch and a valve.

According to still further features in the described preferred embodiments the at least one pharmaceutical agent is dissolved in the in the liquefied polymer.

According to still further features in the described preferred embodiments the at least one pharmaceutical agent is suspended in the liquefied polymer.

According to still further features in the described preferred embodiments the at least one pharmaceutical agent serves for treating at least one disorder in the blood vessel.

According to still further features in the described preferred embodiments the at least one disorder comprises an injury inflicted on tissues of the blood vessel upon implantation of the stent assembly therein.

According to still further features in the described preferred embodiments the at least one disorder is selected from the group consisting of restenosis and in-stent stenosis.

According to still further features in the described preferred embodiments the at least one disorder is hyper cell proliferation.

According to still further features in the described preferred embodiments the at least one coat and the at least one pharmaceutical agent are configured and designed so as to provide a predetermined duration of the delivery.

According to still further features in the described preferred embodiments the delivery is by diffusion.

According to still further features in the described preferred embodiments the delivery is initiated by a radial stretch of the at least one coat, the radial stretch is caused by an expansion of the expensible tubular supporting element.

According to still further features in the described preferred embodiments the at least one coat comprises an inner coat and an outer coat.

According to still further features in the described preferred embodiments the inner coat comprises a layer lining an inner surface of the expensible tubular supporting element.

According to still further features in the described preferred embodiments the outer coat comprises a layer covering an outer surface of the expensible tubular supporting element.

According to still further features in the described preferred embodiments the at least one pharmaceutical agent is constituted by particles embedded in polymer fibers produced during the step of electrospinning.

According to still further features in the described preferred embodiments the step of incorporating at least one pharmaceutical agent into the first coat comprises constituting the at least one pharmaceutical agent into compact objects, and distributing the compact objects between polymer fibers produced during the step of electrospinning.

According to still further features in the described preferred embodiments the compact objects are capsules.

According to still further features in the described preferred embodiments the compact objects are in a powder form.

According to still further features in the described preferred embodiments the distributing of the compact objects is by spraying.

According to still further features in the described preferred embodiments the expensible tubular supporting element comprises a deformable mesh of stainless steel wires.

According to still further features in the described preferred embodiments the coat is of a tubular structure.

According to still further features in the described preferred embodiments the method further comprising mounting the tubular supporting element onto a rotating mandrel.

According to still further features in the described preferred embodiments the method further comprising electrospinning a second liquefied polymer onto the mandrel, hence providing an inner coat.

According to still further features in the described preferred embodiments the method further comprising electrospinning at least one additional liquefied polymer onto the first coat, hence providing at least one additional coat.

According to still further features in the described preferred embodiments the method further comprising providing at least one adhesion layer onto the tubular supporting element.

According to still further features in the described preferred embodiments the method further comprising providing at least one adhesion layer onto at least one coat.

According to still further features in the described preferred embodiments the adhesion layer is an impervious adhesion layer.

According to still further features in the described preferred embodiments the providing at least one adhesion layer is by electrospinning.

According to still further features in the described preferred embodiments the electrospinning step comprises: (i) charging the liquefied polymer thereby producing a charged liquefied polymer; (ii) subjecting the charged liquefied polymer to a first electric field; and (iii) dispensing the charged liquefied polymers within the first electric field in a direction of the mandrel.

According to still further features in the described preferred embodiments the mandrel is of a conductive material.

According to still further features in the described preferred embodiments the first electric field is defined between the mandrel and a dispensing electrode being at a first potential relative to the mandrel.

According to still further features in the described preferred embodiments the method further comprising providing a second electric field defined by a subsidiary electrode being at a second potential relative to the mandrel, the second electric field being for modifying the first electric field.

According to still further features in the described preferred embodiments the subsidiary electrode serves for reducing non-uniformities in the first electric field.

According to still further features in the described preferred embodiments the subsidiary electrode serves for controlling fiber orientation of each of the coats.

According to still further features in the described preferred embodiments the mandrel is of a dielectric material.

According to still further features in the described preferred embodiments the tubular supporting element serves as a mandrel.

According to still further features in the described preferred embodiments the first electric field is defined between the tubular supporting element and a dispensing electrode being at a first potential relative to the tubular supporting element.

According to still further features in the described preferred embodiments the method further comprising providing a second electric field defined by a subsidiary electrode being at a second potential relative to the tubular supporting element, the second electric field being for modifying the first electric field.

According to still further features in the described preferred embodiments the first liquefied polymer is a biocompatible liquefied polymer.

According to still further features in the described preferred embodiments the first liquefied polymer is a biodegradable liquefied polymer.

According to still further features in the described preferred embodiments the first liquefied polymer is a biostable liquefied polymer.

According to still further features in the described preferred embodiments first liquefied polymer is a combination of a biodegradable liquefied polymer and a biostable liquefied polymer.

According to still further features in the described preferred embodiments the second liquefied polymer is a biocompatible liquefied polymer.

According to still further features in the described preferred embodiments the second liquefied polymer is a biodegradable liquefied polymer.

According to still further features in the described preferred embodiments the second liquefied polymer is a biostable liquefied polymer.

According to still further features in the described preferred embodiments the second liquefied polymer is a combination of a biodegradable liquefied polymer and a biostable liquefied polymer.

According to still further features in the described preferred embodiments each of the at least one additional liquefied polymer is independently a biocompatible liquefied polymer.

According to still further features in the described preferred embodiments each of the at least one additional liquefied polymer is independently biodegradable liquefied polymer.

According to still further features in the described preferred embodiments each of the at least one additional liquefied polymer is independently a biostable liquefied polymer.

According to still further features in the described preferred embodiments each of the at least one additional liquefied polymer is independently a combination of a biodegradable liquefied polymer and a biostable liquefied polymer.

According to still further features in the described preferred embodiments the at least one pharmaceutical agent is heparin.

According to still further features in the described preferred embodiments the at least one pharmaceutical agent is a radioactive compound.

According to still further features in the described preferred embodiments the at least one pharmaceutical agent is silver sulfadiazine.

According to still further features in the described preferred embodiments the method further comprising heating the mandrel prior to, during or subsequent to the step of electrospinning.

According to still further features in the described preferred embodiments the heating of the mandrel is selected from the group consisting of external heating and internal heating.

According to still further features in the described preferred embodiments the external heating is by at least one infrared radiator.

According to still further features in the described preferred embodiments the at least one infrared radiator is an infrared lamp.

According to still further features in the described preferred embodiments the internal heating is by a built-in heater.

According to still further features in the described preferred embodiments the built-in heater is an Ohmic built-in heater.

According to still further features in the described preferred embodiments the method further comprising removing the stent assembly from the mandrel.

According to still further features in the described preferred embodiments the method further comprising dipping the stent assembly in a vapor.

According to still further features in the described preferred embodiments the method further comprising heating the vapor.

According to still further features in the described preferred embodiments the vapor is a saturated a DMF vapor.

According to still further features in the described preferred embodiments the method further comprising exposing the stent assembly to a partial vacuum processing.

The present invention successfully addresses the shortcomings of the presently known configurations by providing a stent assembly and a method for manufacturing same, the stent assembly enjoys properties far exceeding those characterizing prior art stent assemblies.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a cross-sectional view of a stent assembly according to the present invention;

FIG. 2a is an end view the stent assembly according to the present invention;

FIG. 2b is an end view of a stent assembly which further comprises at least one adhesion layer, according to the present invention.

FIG. 3 is a tubular supporting element which is designed and constructed for dilating a constricted blood vessel in a body vasculature;

FIG. 4 is a portion of the tubular supporting element comprising a deformable mesh of metal wires;

FIG. 5 is a stent assembly, manufactured according to the teachings of the present invention, occupying a defective site in an artery;

FIG. 6 is a portion of a non-woven web of polymer fibers used to fabricate at least one coat, according to the present invention;

FIG. 7 is a portion of a non-woven web of polymer fibers which comprises a pharmaceutical agent constituted by compact objects and distributed between the electrospun polymer fibers;

FIG. 8 is a is a typical, prior art, electrospinning apparatus;

FIG. 9 is an electrospinning apparatus further including a subsidiary electrode according to the present invention;

FIG. 10 is an electrospinning apparatus including an electrostatic sprayer, two baths and two pumps;

FIG. 11 is an electrospinning apparatus including a supply for holding pharmaceutical agent, an electrostatic sprayer and a conical deflector.

### DESCRIPTION OF THE PREFERRED EMBODIMENTS

The present invention is of a stent assembly which can be used for treating a disorder in a blood vessel. Specifically, the present invention can be used to dilate a constricted blood vessel and to deliver pharmaceutical agent(s) into a body vasculature.

The principles and operation of a stent assembly according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

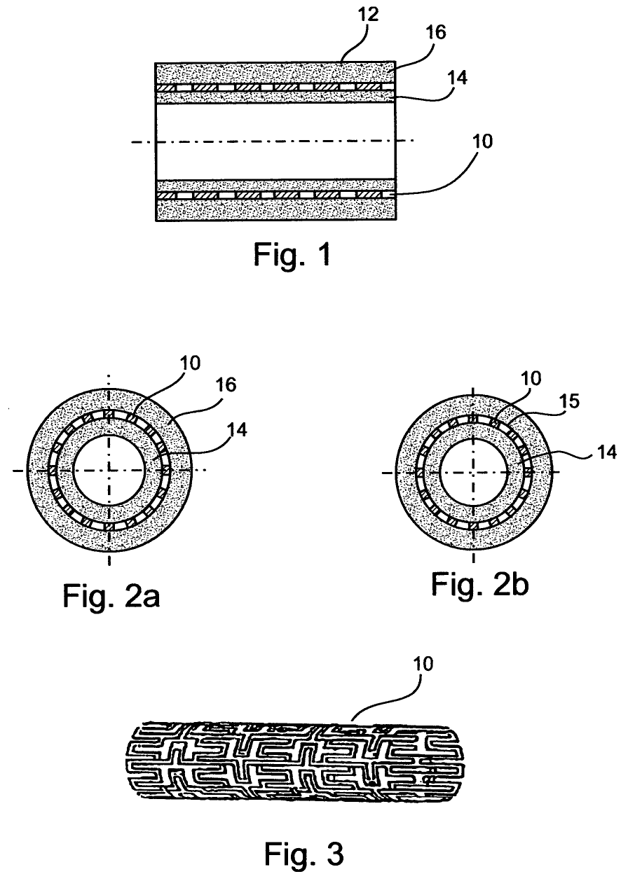

Referring now to the drawings, FIG. 1 illustrates a cross-sectional view of a stent assembly according to a preferred embodiment of the present invention. The stent assembly comprises an expensible tubular supporting element 10 and at least one coat 12, having a predetermined porosity. According to a presently preferred embodiment of the invention, at least one coat 12 comprises an inner coat 14, lining an inner surface of tubular supporting element 10 and an outer coat 16, covering an outer surface of tubular supporting element 10. FIG. 2a illustrates an end view the stent assembly, showing tubular supporting element 10, internally covered by inner coat 14 and externally covered by outer coat 16. Reference is now made to FIG. 2b, illustrating an end view of the stent assembly in which at least one coat 12 further comprises at least one adhesion layer 15, for adhering the components of the stent assembly. A method for providing adhesion layer 15 is further detailed hereinafter.

According to a preferred embodiment of the present invention, at least one of the coats includes at least one pharmaceutical agent incorporated therein for delivery of the pharmaceutical agent into a body vasculature during or after implantation of the stent assembly within the body vasculature. The pharmaceutical agent serves for treating at least one disorder in a blood vessel.

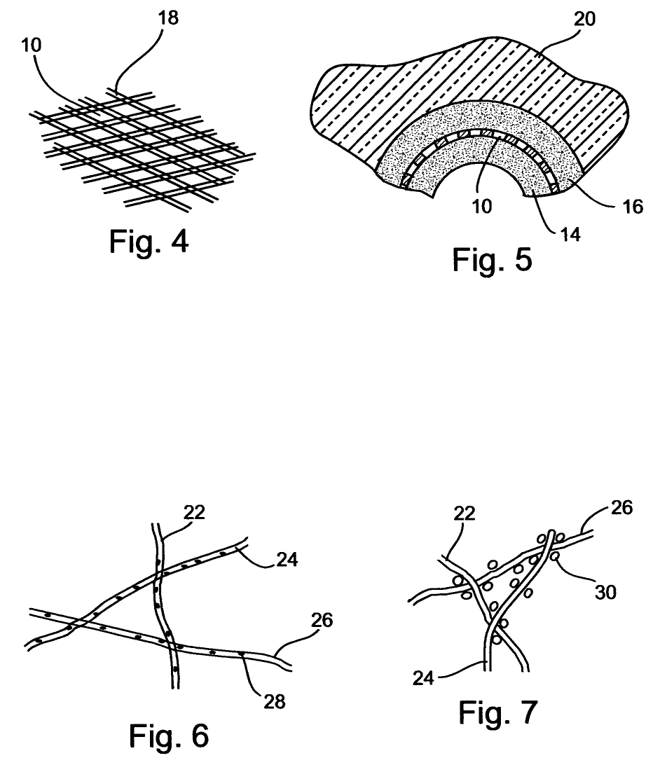

FIG. 3 illustrates tubular supporting element 10 which is designed and constructed for dilating a constricted blood vessel in the body vasculature. Tubular supporting element 10 is operable to expand radially, thereby to dilate a constricted blood vessel. According to a preferred embodiment of the present invention, the expansibility of the stent assembly may be achieved by a suitable construction of tubular supporting element 10 and of at least one coat 12. The construction of tubular supporting element 10 will be described first, with reference to FIG. 4, and the construction of at least one coat 12 will be described thereafter.

Thus, FIG. 4 illustrates a portion of tubular supporting element 10 comprising a deformable mesh of metal wires 18, which can be, for example, a deformable mesh of stainless steel wires. Hence, when the stent assembly is placed in the desired location in an artery, tubular supporting element 10 may be expanded radially, to substantially dilate the arterial tissues surrounding the stent assembly to eradicate a flow constriction in the artery. The expansion may be performed by any method known in the art, for example by using a balloon catheter or by forming tubular supporting element 10 from a material exhibiting temperature-activated shape memory properties, such as Nitinol.

Tubular supporting element 10 is coated by at least one coat 12 which is fabricated from non-woven polymer fibers using an electrospinning method as is further detailed hereinafter. According to a presently preferred embodiment of the invention, the polymer fibers are elastomeric polymer fibers which stretch as tubular supporting element 10 is radially expanded. Referring now again to FIG. 1, in a preferred embodiment of the invention at least one coat 12 comprises inner coat 14 and outer coat 16 both of which are coextensive with the tubular supporting element 10, i.e., tubular supporting element 10 is substantially coated. In other embodiments of the invention, inner coat 14 and/or outer coat 16 may be shorter in length than tubular supporting element 10, in which case at least one end of tubular supporting element 10 is exposed. Still in other embodiments of the invention, inner coat 14 may be absent.

Reference is now made to FIG. 5, which illustrate the stent assembly occupying a defective site 20 in an artery. The outer diameter of the stent assembly in its unexpanded state, including outer coat 16 coating tubular supporting element 10, is such that it ensures transporting of the stent assembly through the artery to defective site 20, for example by a catheter. The expansible range of the stent assembly is such that when in place at defective site 20, the expanded assembly then has a maximum diameter causing the arterial tissues surrounding the stent assembly to be dilated to a degree eradicating the flow constriction at the site.

Implantation of the stent assembly in a blood vessel may result in disorders in the blood vessel, for example an injury inflicted on tissues of the blood vessel upon the implantation, restenosis, in-stent stenosis and hyper cell proliferation. As stated, at least one coat 12 includes at least one pharmaceutical agent incorporated therein for delivery of the pharmaceutical agent into a body vasculature to treat the above disorders. Hence, at least one coat 12 not only serves to graft the assembly to the artery but also functions as a reservoir for storing the pharmaceutical agent to be delivered over a prolonged time period. Within the above diameter limitation, the larger the aggregate volume of at least one coat 12, the larger its capacity to store the pharmaceutical agent.

In addition, inner coat 14 and outer coat 16 are preferably porous so as to accommodate cells migrating from the surrounding tissues and to facilitate the proliferation of these cells.

Reference is now made to FIG. 6 which illustrates a portion of a non-woven web of polymer fibers used to fabricate at least one coat 12. Fibers 22, 24 and 26 intersect and are joined together at the intersections, the resultant interstices rendering the web highly porous. The non-woven web of polymer fibers is produced using an electrospinning process, further described hereinunder, which is capable of producing coatings for forming a graft component having unique advantages. Since electrospun fibers are ultra-thin, they have an exceptionally large surface area, which allows a high quantity of pharmaceutical agent to be incorporated thereon. The surface area of the electrospun polymer fibers approaches that of activated carbon, thereby making the non-woven web of polymer fibers an efficient local drug delivery system. In addition, the porosity of each of inner coat 14 and outer coat 16 can be controlled independently to create evenly distributed pores of predetermined size and orientation for promoting a high degree of tissue ingrowth and cell endothelization.

The preferred mechanism of pharmaceutical agent release from at least one coat 12 is by diffusion, regardless of the technique employed to embed the pharmaceutical agent therein. The duration of therapeutic drug release in a predetermined concentration depends on several variants, which may be controlled during the manufacturing process. One variant is the chemical nature of the carrier polymer and the chemical means binding the pharmaceutical agent to it. This variant may be controlled by a suitable choice of the polymer(s) used in the electrospinning process. Another variant is the area of contact between the body and the pharmaceutical agent, which can be controlled by varying the free surface of the electrospun polymer fibers. Also affecting the duration of pharmaceutical agent release is the method used to incorporate the pharmaceutical agent within at least one coat 12, as is further described herein.

According to a preferred embodiment of the present invention, at least one coat 12 includes a number of sub-layers. As a function of their destination, the sub-layers can be differentiated, by fiber orientation, polymer type, pharmaceutical agent incorporated therein, and desired release rate thereof. Thus, pharmaceutical agent release during the first hours and days following implantation may be achieved by incorporating a solid solution, containing a pharmaceutical agent such as anticoagulants and antithrombogenic agents, in a sub-layer of readily soluble biodegradable polymer fibers. Thus, during the first period following implantation the pharmaceutical agent that releases includes anticoagulants and antithrombogenic agents.

Referring now again to FIG. 6, the pharmaceutical agent may be constituted by particles 28 embedded in the electrospun polymer fibers forming a sub-layer of at least one coat 12. This method is useful for pharmaceutical agent release during the first post-operative days and weeks. To this end, the pharmaceutical agent can include antimicrobials or antibiotics, thrombolytics, vasodilators, and the like. The duration of the delivery process is effected by the type of polymer used for fabricating the corresponding sub-layer. Specifically, optimal release rate is ensured by using moderately stable biodegradable polymers.

Reference is now made to FIG. 7, which illustrates an alternative method for incorporating the pharmaceutical agent in at least one coat 12, ensuring pharmaceutical agent release during the first post-operative days and weeks. Thus, according to a preferred embodiment of the present invention, the pharmaceutical agent is constituted by compact objects 30 distributed between the electrospun polymer fibers of at least one coat 12. In a presently preferred embodiment of the invention, compact objects 30 may be in any known form, such as, but not limited to, moderately stable biodegradable polymer capsules.

The present invention is also capable of providing release of the pharmaceutical agent, which may last from several months to several years. According to this embodiment of the present invention, the pharmaceutical agent is dissolved or encapsulated in a sub-layer made of biosatable fibers. The rate diffusion from within a biostable sub-layer is substantially slower, thereby ensuring a prolonged effect of pharmaceutical agent release. Pharmaceutical agent suitable for such prolonged release include for example, antiplatelets, growth-factor antagonists and free radical scavengers.

Thus, the sequence of pharmaceutical agent release and impact longevity of a certain specific pharmaceutical agents is determined by the type of drug-incorporated polymer, the method in which the pharmaceutical agent is introduced into the electrospun polymer fibers, the sequence of layers forming at least one coat 12, the matrix morphological peculiarities of each layer and by pharmaceutical agent concentration.

These key factors are controlled by the electrospinning method of manufacturing described herein. Although electrospinning can be efficiently used for generating large diameter shells, the nature of the electrospinning process prevents efficient generation of products having small diameters, such as a medicated, polymer-coated stent assembly. In particular, electrospinning manufacturing of small diameter coats result in predominant axial orientation of the fibers leading to a considerable predominance of an axial over radial strength.

While reducing the present invention to practice, it was uncovered that improved mechanical strength of the coating can be achieved when substantially thick and strong fibers are situated axially, and substantially thin and highly elastic fibers are situated in a transverse (polar) direction.

Thus, according to the present invention there is provided a method of producing a stent assembly, the method comprising electrospinning a first liquefied polymer onto expensible tubular supporting element 10, thereby coating tubular supporting element 10 with a first coat having a predetermined porosity; and incorporating at least one pharmaceutical agent into the first coat. As stated, in some embodiments the pharmaceutical agent is mixed with the liquefied polymer prior to the electrospinning process, hence the step of incorporating the pharmaceutical agent into the first coat is concomitant with the step of electrospinning.

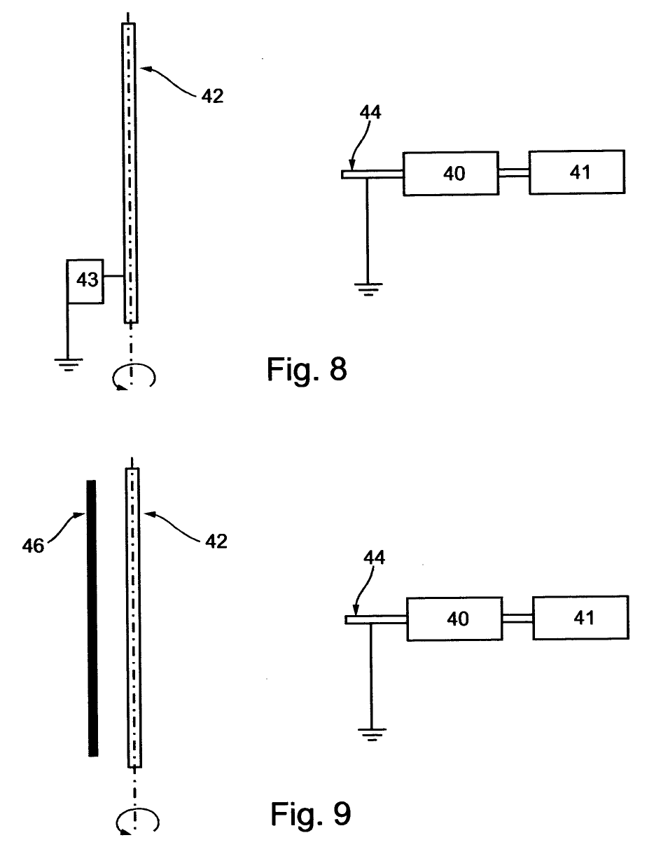

The electrospinning steps may be performed using any electrospinning apparatus known in the art. Referring now again to the drawings, FIG. 8 illustrate a typical electrospinning apparatus, which includes a pump 40, a mandrel 42 connected to a power supply 43 and a dispensing electrode 44. Pump 40 is connected to a bath 41 and serves for drawing the liquid polymer stored in bath 41 through a syringe (not shown in FIG. 8) into dispensing electrode 44. Mandrel 42 and dispensing electrode 44 are held under a first potential difference, hence generating a first electric field therebetween. According to the electrospinning method, liquefied polymer is drawn into dispensing electrode 44, and then, subjected to the first electric field, charged and dispensed in a direction of mandrel 42. Moving with high velocity in the inter-electrode space, jet of liquefied polymer cools or solvent therein evaporates, thus forming fibers which are collected on the surface of mandrel 42.

Reference is now made to FIG. 9, which depicts an electrospinning apparatus used according to another preferred embodiment of the present invention in the manufacturing of the stent assembly. Hence, the method may further comprise providing a second electric field defined by a subsidiary electrode 46 which is kept at a second potential difference relative to mandrel 42. The purpose of the second electric field (and of the subsidiary electrode 46) is to modify the first electric field, so as to ensure a predetermined fiber orientation while forming the coat. Such predetermined orientation is important, in order to provide a stent assembly combining the above structural characteristics.

There are two alternatives for providing outer coat 16 of tubular supporting element 10. The first is to mount tubular supporting element 10 on mandrel 42, prior to the electrospinning process, and the second is to use tubular supporting element 10 as a mandrel.

In the preferred embodiment in which mandrel 42 is used as a carrier for tubular supporting element 10, mandrel 42 may function as a metal electrode to which a high voltage is applied to establish the electric field. As a consequence, the polymer fibers emerging from dispensing electrode 44 are projected toward mandrel 42 and form outer coat 16 on tubular supporting element 10. This coating covers both gaps between the metal wires and said metal wires of tubular supporting element 10.

In other embodiments, outer coat 16 exposes the gaps between the metal wires and exclusively covers metal wires of tubular supporting element 10. This may be achieved either by using tubular supporting element 10 as a mandrel, or by using a dielectric material mandrel, as opposed to a conductive mandrel. Hence, according to this embodiment of the invention the metal mesh of tubular supporting element 10 serves as an electrode to be connected to a source of high voltage to establish an electrostatic field which extends to the stent but not to the mandrel (in the preferred embodiments in which an isolating mandrel is used). Thus, polymer fibers are exclusively attracted to the wires of tubular supporting element 10 exposing the gaps therebetween. In any case, the resultant polymer-coated stent therefore has pores which serve for facilitating pharmaceutical agent delivery from the stent assembly into body vasculature.

According to a preferred embodiment of the present invention the method further comprising providing inner coat 14 which lines the inner surface of tubular supporting element 10. Hence, according to a presently preferred embodiment of the invention, the electrospinning process is first employed so as to directly coat mandrel 42, thereby to provide inner coat 14. Once mandrel 42 is coated, the electrospinning process is temporarily ceased and tubular supporting element 10 is slipped onto the mandrel and drawn over inner coat 14. Outer coat 16 is then provided by resuming the electrospinning process onto tubular supporting element 10.

Since the operation providing inner coat 14 demands a process cessation for a certain period, a majority of solvent contained in inner coat 14 may be evaporated. This may lead to a poor adhesion between the components of the stent assembly, once the process is resumed, and might result in the coating stratification following stent graft opening.

The present invention successfully addresses the above-indicated limitation by two optimized techniques. According to one technique, the outer sub-layer of inner coat 14 and the inner sub-layer of outer coat 16 are each made by electrospinning with upgraded capacity. A typical upgrading can may range from about 50% to about 100%. This procedure produce a dense adhesion layer made of thicker fibers with markedly increased solvent content. A typical thickness of the adhesion layer ranges between about 20 μm and about 30 μm, which is small compared to the overall diameter of the stent assembly hence does not produce considerable effect on the coats general parameters. According to an alternative technique, the adhesion layer comprises an alternative polymer with lower molecular weight than the major polymer, possessing high elastic properties and reactivity.

Other techniques for improving adhesion between the layers and tubular supporting element 10 may also be employed. For example, implementation of various adhesives, primers, welding, chemical binding in the solvent fumes can be used. Examples for suitable materials are silanes such as aminoethyaminopropyl-triacytoxysilane and the like.

The advantage of using the electrospinning method for fabricating at least one coat 12 is flexibility of choosing the polymer types and fibers thickness, thereby providing a final product having the required combination of strength, elastic and other properties as delineated herein. In addition, an alternating sequence of the sub-layers forming at least one coat 12, each made of differently oriented fibers, determines the porosity distribution nature along the stent assembly wall thickness. Still in addition, the electrospinning method has the advantage of allowing the incorporation of various chemical components, such as pharmaceutical agents, to be incorporated in the fibers by mixing the pharmaceutical agents in the liquefied polymers prior to electrospinning.

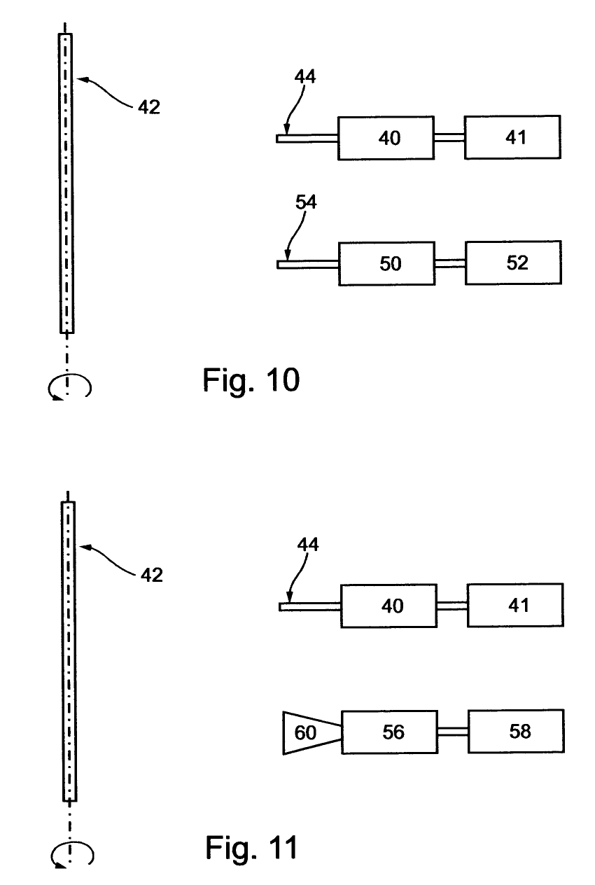

Reference is now made to FIG. 10, which depicts an electrospinning apparatus used according to another preferred embodiment of the present invention in the manufacturing of the stent assembly. In a presently preferred embodiment of the invention, the pharmaceutical agent is mixed with the liquefied polymer in bath 52 prior to the step of electrospinning. Then, the obtained compound is supplied by a pump 50 to an electrostatic sprayer 54 to be sprayed onto tubular supporting element 10 (not shown in FIG. 10) which is mounted on mandrel 42. Preferably, axially oriented fibers, which do not essentially contribute to the radial strength properties, can be made of biodegradable polymer and be drug-loaded. Such incorporation of the pharmaceutical agent results in slow release of the agent upon biodegradation of the fibers. The mixing of the pharmaceutical agent in the liquefied polymer may be done using any suitable method, for example by dissolving or suspending. The pharmaceutical agent may be constituted by particles or it may be in a dissolved form.

In the preferred embodiments in which the pharmaceutical agent is to be entrapped in the interstices of the non-woven web at least one coat 12, the agent is preferably in a powder form or micro-encapsulated particulates form so that it can be sprayed as a shower of particles onto a specific layer of at least one coat 12, once formed.

Reference is now made to FIG. 11 which depicts electrospinning apparatus used according to a presently preferred embodiment of the present invention. A biocompatible pharmaceutical agent drawn from a supply 58 is fed to electrostatic sprayer 56, whose output is sprayed through a conical deflector 60 to yield a spray of pharmaceutical particles which are directed toward the stent assembly.

It should be understood, that although the invention has been described in conjunction with tubular supporting element 10, other medical implants, not necessarily of tubular structure, may be coated using the techniques of the present invention. For example, grafts and patches, which may be coated prior to procedure of implantation or application can be drug-loaded and enjoy the advantages as described herein.

The at least one coat 12 may be made from any known biocompatible polymer. In the layers which incorporate pharmaceutical agent, the polymer fibers are preferably a combination of a biodegradable polymer and a biostable polymer.

The list of biostable polymers with a relatively low chronic tissue response include polycarbonate based aliphatic polyurethanes, siloxane based aromatic polyurethanes, polydimethylsiloxane and other silicone rubbers, polyester, polyolefins, polymethyl-methacrylate, vinyl halide polymer and copolymers, polyvinyl aromatics, polyvinyl esters, polyamides, polyimides, polyethers and many others that can be dissolved in appropriate solvents and electrically spun on the stent.

Biodegradable fiber-forming polymers that can be used include poly (L-lactic acid), poly (lactide-co-glycolide), polycaprolactone, polyphosphate ester, poly (hydroxy-butyrate), poly (glycolic acid), poly (DL-lactic acid), poly (amino acid), cyanocrylate, some copolymers and biomolecules such as DNA, silk, chitozan and cellulose.

These hydrophilic and hydrophobic polymers which are readily degraded by microorganisms and enzymes are suitable for encapsulating material for drugs. In particular, Polycaprolacton has a slower degradation rate than most other polymers and is therefore especially suitable for controlled-release of pharmaceutical agent over long periods of time scale ranging from about 2 years to about 3 years.

Suitable pharmaceutical agents that can be incorporated in at least one coat 12 include heparin, tridodecylmethylammonium-heparin, epothilone A, epothilone B, rotomycine, ticlopidine, dexamethasone, caumadin, and other pharmaceuticals falling generally into the categories of antithrombotic drugs, estrogens, corticosteroids, cytostatics, anticoagulant drugs, vasodilators, and antiplatelet drugs, trombolytics, antimicrobials or antibiotics, antimitotics, antiproliferatives, antisecretory agents, nonsterodial antiflammentory drugs, grow factor antagonists, free radical scavengers, antioxidants, radiopaque agents, immunosuppressive agents and radio-labeled agents.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials, Devices and Methods

A Carbothane PC-3575A was purchased from Thermedics Polymer Products, and was used for coating. This polymer has satisfactory fiber-generation abilities, it is biocompatibility and is capable of lipophilic drug incorporation. A mixture of dimethylformamide and toluene of ratio ranging from 1:1 to 1:2 was used as a solvent in all experiments.

A PHD 2000 syringe pump was purchased from Harvard Apparatus and was used in the electrospinning apparatus. A spinneret, 0.9 mm in inner diameter, was used as the dispensing electrode. The flow-rate of the spinneret was between 0.05 ml/min and 5 ml/min. The dispensing electrode was grounded while the mandrel was kept at a potential of about 20-50 kV. The mandrel, made of polished stainless steel, was rotated at frequency of 100-150 rotations per minute.

The dispensing electrode was positioned about 25 cm to 35 cm from the precipitation electrode and was connected to the pump with flexible polytetrafluorethylene tubes. Reciprocal motion of the dispensing electrode, 30-40 mm in amplitude, was enabled along the mandrel longitudinal axis at a frequency of 2-3 motions/min.

### Example 1

A stent assembly, 16 mm in length was manufactured using a stainless-steel stent, 3 mm in diameter in its expanded state, 1.9 mm in diameter in its non-expanded state, as the tubular supporting element. The used stainless-steel stent is typically intended for catheter and balloon angioplasty. For adhesion upgrading in polymer coating, the stent was exposed to 160-180 kJ/m² corona discharge, rinsed by ethyl alcohol and deionized water, and dried in a nitrogen flow. The concentration of the solution was 8%; the viscosity was 560 cP; and the conductivity 0.8 μS. For the pharmaceutical agent, heparin in tetrahydrofurane solution was used, at a concentration of 250 U/ml. The polymer to heparin-solution ratio was 100:1. A metal rod, 1.8 mm in diameter and 100 mm in length was used as a mandrel.

To ensure uniform, high-quality coating of an electrode having a low curvature radius, a planar subsidiary electrode was positioned near the mandrel, at a 40 mm distance from the longitudinal axis of the mandrel. The subsidiary electrode potential and the mandrel potential were substantially equal.

A two step coating process was employed. First, the mandrel was coated by electrospinning with polymer fiber layer the thickness of which was about 40 μm. Once the first step was accomplished, the tubular supporting element was put over the first coat hence an inner coating for the tubular supporting element was obtained. Secondly, an outer coating was applied to the outer surface of the tubular supporting element. The thickness of the outer coat was about 100 μm.

The stent assembly was removed from the mandrel, and was placed for about 30 seconds into the saturated DMF vapor atmosphere at 45° C., so as to ensure upgrading the adhesion strength between the inner coat and the outer coat. Finally, to remove solvent remnants, the stent was exposed to partial vacuum processing for about 24 hours.

### Example 2

A stent assembly was manufactured as described in Example 1, however the pharmaceutical agent was a heparin solution at a concentration of 380 U/ml mixed with 15% poly (DL-Lactide-CD-Glycolide) solution in chloroform.

In addition, for the dispensing electrode, two simultaneously operating spinnerets were used, mounted one above the other with a height difference of 20 mm therebetween. The first operable to dispense polyurethane while the second operable to dispense the biodegradable polymer poly (L-lactic acid). To ensure desirable correlation between the fiber volumes of polyurethane and the biodegradable polymer, the solution feeding were 0.1 ml/min for the first spinneret and 0.03 ml/min for the second spinneret.

### Example 3

A stent assembly was manufactured from the materials described in Example 1.

A two step coating process was employed. First, the mandrel was coated by electrospinning with polymer fiber layer the thickness of which was about 60 μm. Once the first step was accomplished, the tubular supporting element was put over the first coat, hence an inner coating for the tubular supporting element was obtained. Before providing the outer coat, a subsidiary electrode, manufactured as a ring 120 mm in diameter, was mounted 16 mm behind the mandrel.

The subsidiary electrode was made of a wire 1 mm in thickness. The plane engaged by the subsidiary electrode was perpendicular to the mandrel's longitudinal axis. As in Example 1, the subsidiary electrode potential and the mandrel potential were substantially equal, however, unlike Example 1, the subsidiary electrode was kinematically connected to the spinneret, so as to allow synchronized motion of the two.

The second coat was applied as in Example 1, until an overall thickness of 100 μm for the coatings was achieved.

Tests have shown that the fibers of biodegradable heparin-loaded polymer have predominant orientation, coinciding with the mandrel longitudinal axis, whereas the polyurethane fibers have predominant transverse (polar) orientation.

### Example 4

A stent assembly was manufactured as described in Example 1, with an aspirin powder added to the polymer solution. The particle root-mean-square (RMS) diameter was 0.2 μm. The powder mass content in the solution in terms of dry polymer amounted to 3.2%. For obtaining stable suspension, the composition was mixed for 6 hours using a magnetic stirrer purchased from Freed electric with periodic (1:60) exposure to a 32 Khz ultrasound obtained using a PUC40 device.

### Example 5

A stent assembly was manufactured as described under Example 3, yet the viscosity of the solution employed was higher (770 cP), so was its conductivity (2 μS). A solution having these characteristics promotes the production of coarser fibers and a flimsier fabric.

In addition, an aspirin powder was conveyed to a fluidized bed and fed to the spinneret. Sputtering and electrospinning were simultaneous but in an interrupted mode: 5 second sputtering followed by a 60 seconds break. The potential difference between the dispensing electrode and the mandrel was 23 kV, the interelectrode separation was 15 cm, and powder feeding rate was 100 mg/min.

### Example 6

A stent assembly having an outer coat and an inner coat was manufactured as described herein. The outer coat was made of a polymer solution having the parameters specified in Example 4, only a heparin solution was added thereto, as described in Example 3. The stent inner coating was made of polymer solution with the parameters specified in Example 1, only a heparin solution was added thereto, as described in Example 3. Thus, the inner coating was characterized by thin fibers and pore size of about 1 μm. A coating of this character ensures efficient surface endothelization. The outer surface had pores size of about 5-15 μm to ensure the ingrowth of tissues.

### Example 7

A stent assembly was manufactured as described in Example 1, except that for both inner coat and outer coat a 6% ratamycine solution in chloroform was used instead of heparin.

### Example 8

A stent assembly was manufactured as described in Example 1, except that a ticlopidine solution in chloroform was used instead of a heparin solution for the outer coat, whereas the inner coat was manufactured as in Example 1.

### Example 9

A stent assembly was manufactured from the materials described in Example 1, however, before coating by electrospinning the stent was first dipped into a TECOFLEX Adhesive 1-MP solution. In addition, the distance between the mandrel and subsidiary electrode was reduced to 20 mm. Still in addition, the step of post-treatment in solvent vapor was omitted.

The purpose of the present example was to generate an outer coat which exposes the gaps between the metal wires and exclusively covers metal wires of tubular supporting element. Hence, the mandrel was made of a dielectric material, whereas the tubular supporting element was kept under a potential of 25 kV, via electrical contacts.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admthat such reference is available as prior art to the present invention.

What is claimed is:

1. A stent assembly comprising an expensible tubular supporting element and at least one coat of electrospun polymer fibers, each of said at least one coat having a predetermined porosity, said at least one coat including at least one pharmaceutical agent incorporated therein for delivery of said at least one pharmaceutical agent into a body vasculature during or after implantation of the stent assembly within said body vasculature.

2. The stent assembly of claim 1, wherein said expensible tubular supporting element is designed and constructed for dilating a constricted blood vessel in said body vasculature.

3. The stent assembly of claim 1, wherein each of said at least one coat is independently a tubular structure.

4. The stent assembly of claim 2, wherein said at least one pharmaceutical agent serves for treating at least one disorder in said blood vessel.

5. The stent assembly of claim 4, wherein said at least one disorder comprises an injury inflicted on tissues of said blood vessel upon implantation of the stent assembly therein.

6. The stent assembly of claim 4, wherein said at least one disorder is selected from the group consisting of restenosis and in-stent stenosis.

7. The stent assembly of claim 4, wherein said at least one disorder is hyper cell proliferation.

8. The stent assembly of claim 1, wherein said at least one coat and said at least one pharmaceutical agent are configured and designed so as to provide a predetermined sustained release rate for effecting said delivery.

9. The stent assembly of claim 1, wherein said at least one coat and said at least one pharmaceutical agent are configured and designed so as to provide a predetermined duration of said delivery.

10. The stent assembly of claim 1, wherein said delivery is by diffusion.

11. The stent assembly of claim 10, wherein said delivery is initiated by a radial stretch of said at least one coat, said radial stretch is caused by an expansion of said expensible tubular supporting element.

12. The stent assembly of claim 1, wherein said expensible tubular supporting element comprises a deformable mesh of metal wires.

13. The stent assembly of claim 1, wherein said expensible tubular supporting element comprises a deformable mesh of stainless steel wires.

14. The stent assembly of claim 1, wherein said at least one coat comprises an inner coat and an outer coat.

15. The stent assembly of claim 14, wherein said inner coat comprises a layer lining an inner surface of said expensible tubular supporting element.

16. The stent assembly of claim 14, wherein said outer coat comprises a layer covering an outer surface of said expansible tubular supporting element.

17. The stent assembly of claim 1, wherein said electrospun polymer fibers are made of a biocompatible polymer.

18. The stent assembly of claim 1, wherein at least a portion of said electrospun polymer fibers is made of a biodegradable polymer.

19. The stent assembly of claim 1, wherein at least a portion of said electrospun polymer fibers is made of a biostable polymer.

20. The stent assembly of claim 1, wherein at least a portion of said electrospun polymer fibers is made of a combination of a biodegradable polymer and a biostable polymer.

21. The stent assembly of claim 1, wherein said electrospun polymer fibers are manufactured from a liquefied polymer.

22. The stent assembly of claim 21, wherein said at least one pharmaceutical agent is dissolved in said liquefied polymer.

23. The stent assembly of claim 21, wherein said at least one pharmaceutical agent is suspended in said liquefied polymer.

24. The stent assembly of claim 1, wherein said at least one pharmaceutical agent is constituted by compact objects distributed between said electrospun polymer fibers of said at least one coat.

25. The stent assembly of claim 24, wherein said compact objects are capsules.

26. The stent assembly of claim 1, wherein said at least one pharmaceutical agent is constituted by particles embedded in said electrospun polymer fibers.

27. The stent assembly of claim 1, wherein said at least one coat includes an adhesion layer.

28. The stent assembly of claim 27, wherein said adhesion layer is impervious adhesion layer.

29. The stent assembly of claim 27, wherein said adhesion layer is formed from electrospun polymer fibers.

30. The stent assembly of claim 1, wherein said electrospun polymer fibers are selected from the group consisting of polyethylene-terephtalat fibers and polyurethane fibers.

31. The stent assembly of claim 1, wherein said at least one pharmaceutical agent comprises heparin or heparin derivative.

32. The stent assembly of claim 1, wherein said at least one pharmaceutical agent comprises a radioactive compound.

33. The stent assembly of claim 1, wherein said at least one pharmaceutical agent comprises silver sulfadiazine.

34. The stent assembly of claim 1, wherein said at least one pharmaceutical agent comprises an antiproliferative drug.

35. The stent assembly of claim 1, wherein said at least one pharmaceutical agent comprises an anticoagulant drug.

36. The stent assembly of claim 12, wherein said at least one coat exposes gaps between said metal wires and exclusively covers said metal wires.

37. The stent assembly of claim 12, wherein said at least one coat substantially covers both gaps between said metal wires and said metal wires.

38. A method of producing a stent assembly, the method comprising:
(a) electrospinning a first liquefied polymer onto an expensible tubular supporting element, thereby coating said tubular supporting element with a first coat having a predetermined porosity; and
(b) incorporating at least one pharmaceutical agent into said first coat.

39. The method of claim 38, wherein said at least one pharmaceutical agent is mixed with said liquefied polymer prior to said step of electrospinning, hence said step of incorporating said at least one pharmaceutical agent into said first coat is concomitant with said electrospinning.

40. The method of claim 39, wherein said at least one pharmaceutical agent is dissolved in said in said liquefied polymer.

41. The method of claim 39, wherein said at least one pharmaceutical agent is suspended in said liquefied polymer.

42. The method of claim 39, wherein said at least one pharmaceutical agent is constituted by particles embedded in polymer fibers produced during said step of electrospinning.

43. The method of claim 38, wherein said step of incorporating at least one pharmaceutical agent into said first coat comprises constituting said at least one pharmaceutical agent into compact objects, and distributing said compact objects between polymer fibers produced during said step of electrospinning.

44. The method of claim 43, wherein said compact objects are capsules.

45. The method of claim 43, wherein said compact objects are in a powder form.

46. The method of claim 43, wherein said distributing of said compact objects is by spraying.

47. The method of claim 38, wherein said expensible tubular supporting element comprises a deformable mesh of metal wires.

48. The method of claim 38, wherein said expensible tubular supporting element comprises a deformable mesh of stainless steel wires.

49. The method of claim 38, wherein said coat is of a tubular structure.

50. The method of claim 38, further comprising mounting said tubular supporting element onto a rotating mandrel, prior to said step (a).

51. The method of claim 50, further comprising electrospinning a second liquefied polymer onto said mandrel, prior to said step (a), hence providing an inner coat.

52. The method of claim 38, further comprising electrospinning at least one additional liquefied polymer onto said first coat, hence providing at least one additional coat.

53. The method of claim 38, further comprising providing at least one adhesion layer onto said tubular supporting element.

54. The method of claim 51, further comprising providing at least one adhesion layer onto at least one coat.

55. The method of claim 53, wherein said adhesion layer is an impervious adhesion layer.

56. The method of claim 54, wherein said adhesion layer is an impervious adhesion layer.

57. The method of claim 53, wherein said providing at least one adhesion layer is by electrospinning.

58. The method of claim 54, wherein said providing at least one adhesion layer is by electrospinning.

59. The method of claim 50, wherein said electrospinning step comprises:
(i) charging said liquefied polymer thereby producing a charged liquefied polymer;
(ii) subjecting said charged liquefied polymer to a first electric field; and
(iii) dispensing said charged liquefied polymers within said first electric field in a direction of said mandrel.

60. The method of claim 59, wherein said mandrel is of a conductive material.

61. The method of claim 60, wherein said first electric field is defined between said mandrel and a dispensing electrode being at a first potential relative to said mandrel.

62. The method of claim 60, further comprising providing a second electric field defined by a subsidiary electrode being at a second potential relative to said mandrel, said second electric field being for modifying said first electric field.

63. The method of claim 62, wherein said subsidiary electrode serves for reducing non-uniformities in said first electric field.

64. The method of claim 62, wherein said subsidiary electrode serves for controlling fiber orientation of each of said coats.

65. The method of claim 59, wherein said mandrel is of a dielectric material.

66. The method of claim 59, wherein said tubular supporting element serves as a mandrel.

67. The method of claim 65, wherein said first electric field is defined between said tubular supporting element and a dispensing electrode being at a first potential relative to said tubular supporting element.

68. The method of claim 65, further comprising providing a second electric field defined by a subsidiary electrode being at a second potential relative to said tubular supporting element, said second electric field being for modifying said first electric field.

69. The method of claim 68, wherein said subsidiary electrode serves for reducing non-uniformities in said first electric field.

70. The method of claim 68, wherein said subsidiary electrode serves for controlling fiber orientation of each of said coats.

71. The method of claim 38, wherein said first liquefied polymer is a biocompatible liquefied polymer.

72. The method of claim 38, wherein said first liquefied polymer is a biodegradable liquefied polymer.

73. The method of claim 38, wherein said first liquefied polymer is a biostable liquefied polymer.

74. The method of claim 38, wherein first liquefied polymer is a combination of a biodegradable liquefied polymer and a biostable liquefied polymer.

75. The method of claim 51, wherein said second liquefied polymer is a biocompatible liquefied polymer.

76. The method of claim 51, wherein said second liquefied polymer is a biodegradable liquefied polymer.

77. The method of claim 51, wherein said second liquefied polymer is a biostable liquefied polymer.

78. The method of claim 51, wherein said second liquefied polymer is a combination of a biodegradable liquefied polymer and a biostable liquefied polymer.

79. The method of claim 52, wherein each of said at least one additional liquefied polymer is independently a biocompatible liquefied polymer.

80. The method of claim 52, wherein each of said at least one additional liquefied polymer is independently biodegradable liquefied polymer.

81. The method of claim 52, wherein each of said at least one additional liquefied polymer is independently a biostable liquefied polymer.

82. The method of claim 52, wherein each of said at least one additional liquefied polymer is independently a combination of a biodegradable liquefied polymer and a biostable liquefied polymer.

83. The method of claim 38, wherein said at least one pharmaceutical agent is heparin.

84. The method of claim 38, wherein said at least one pharmaceutical agent is a radioactive compound.

85. The method of claim 38, wherein said at least one pharmaceutical agent is silver sulfadiazine.

86. The method of claim 50, further comprising heating said mandrel prior to, during or subsequent to said step of electrospinning.

87. The method of claim 86, wherein said heating of said mandrel is selected from the group consisting of external heating and internal heating.

88. The method of claim 87, wherein said external heating is by at least one infrared radiator.

89. The method of claim 88, wherein said at least one infrared radiator is an infrared lamp.

90. The method of claim 87, wherein said internal heating is by a built-in heater.

91. The method of claim 90, wherein said built-in heater is an Ohmic built-in heater.

92. The method of claim 50, further comprising removing the stent assembly from said mandrel.

93. The method of claim 92, further comprising dipping the stent assembly in a vapor.

94. The method of claim 93, further comprising heating said vapor.

95. The method of claim 92, wherein said vapor is saturated a DMF vapor.

96. The method of claim 38, further comprising exposing the stent assembly to a partial vacuum processing.

97. A method of treating a constricted blood vessel, the method comprising placing a stent assembly in the constricted blood vessel, said stent assembly comprises an expensible tubular supporting element and at least one coat of electrospun polymer fibers, each of said at least one coat having a predetermined porosity, said at least one coat including at least one pharmaceutical agent incorporated therein for delivery of said at least one pharmaceutical agent into a body vasculature during or after implantation of the stent assembly within said body vasculature.

98. The method of claim 97, wherein said expensible tubular supporting element is designed and constructed for dilating a constricted blood vessel in said body vasculature.

99. The method of claim 97, wherein each of said at least one coat is independently a tubular structure.

100. The method of claim 98, wherein said at least one pharmaceutical agent serves for treating at least one disorder in said blood vessel.

101. The method of claim 100, wherein said at least one disorder comprises an injury inflicted on tissues of said blood vessel upon implantation of the stent assembly therein.

102. The method of claim 100, wherein said at least one disorder is selected from the group consisting of restenosis and in-stent stenosis.

103. The method of claim 100, wherein said at least one disorder is hyper cell proliferation.

104. The method of claim 97, wherein said at least one coat and said at least one pharmaceutical agent are configured and designed so as to provide a predetermined sustained release rate for effecting said delivery.

105. The method of claim 97, wherein said at least one coat and said at least one pharmaceutical agent are configured and designed so as to provide a predetermined duration of said delivery.

106. The method of claim 97, wherein said delivery is by diffusion.

107. The method of claim 106, wherein said delivery is initiated by a radial stretch of said at least one coat, said radial stretch is caused by an expansion of said expensible tubular supporting element.

108. The method of claim 97, wherein said expensible tubular supporting element comprises a deformable mesh of metal wires.

109. The method of claim 97, wherein said expensible tubular supporting element comprises a deformable mesh of stainless steel wires.

110. The method of claim 97, wherein said at least one coat comprises an inner coat and an outer coat.

111. The method of claim 110, wherein said inner coat comprises a layer lining an inner surface of said expansible tubular supporting element.

112. The method of claim 110, wherein said outer coat comprises a layer covering an outer surface of said expensible tubular supporting element.

113. The method of claim 97, wherein said electrospun polymer fibers are made of a biocompatible polymer.

114. The method of claim 97, wherein at least a portion of said electrospun polymer fibers is made of a biodegradable polymer.

115. The method of claim 97, wherein at least a portion of said electrospun polymer fibers is made of a biostable polymer.

116. The method of claim 97, wherein at least a portion of said electrospun polymer fibers is made of a combination of a biodegradable polymer and a biostable polymer.

117. The method of claim 97, wherein said electrospun polymer fibers are manufactured from a liquefied polymer.

118. The method of claim 117, wherein said at least one pharmaceutical agent is dissolved in said liquefied polymer.

119. The method of claim 117, wherein said at least one pharmaceutical agent is suspended in said liquefied polymer.

120. The method of claim 97, wherein said at least one pharmaceutical agent is constituted by compact objects distributed between said electrospun polymer fibers of said at least one coat.

121. The method of claim 120, wherein said compact objects are capsules.

122. The method of claim 97, wherein said at least one pharmaceutical agent is constituted by particles embedded in said electrospun polymer fibers.

123. The method of claim 97, wherein said at least one coat includes an adhesion layer.

124. The method of claim 123, wherein said adhesion layer is impervious adhesion layer.

125. The method of claim 123, wherein said adhesion layer is formed from electrospun polymer fibers.

126. The method of claim 97, wherein said electrospun polymer fibers are selected from the group consisting of polyethylene-terephtalat fibers and polyurethane fibers.

127. The method of claim 97, wherein said at least one pharmaceutical agent comprises heparin or heparin derivative.

128. The method of claim 97, wherein said at least one pharmaceutical agent comprises a radioactive compound.

129. The method of claim 97, wherein said at least one pharmaceutical agent comprises silver sulfadiazine.

130. The method of claim 97, wherein said at least one pharmaceutical agent comprises an antiproliferative drug.

131. The method of claim 97, wherein said at least one pharmaceutical agent comprises an anticoagulant drug.

132. The method of claim 108, wherein said at least one coat exposes gaps between said metal wires and exclusively covers said metal wires.

133. The method of claim 108, wherein said at least one coat substantially covers both gaps between said metal wires and said metal wires.

134. A method of dilating a constricted blood vessel, the method comprising:
(a) providing a stent assembly comprises an expensible tubular supporting element and at least one coat of electrospun polymer fibers, each of said at least one coat having a predetermined porosity, said at least one coat including at least one pharmaceutical agent incorporated therein;
(b) placing said stent assembly to a constricted region in the constricted blood vessel; and
(c) radially expanding said stent assembly within the blood vessel so as to dilate said constricted region and to allow blood flow through the blood vessel.

135. The method of claim 134, wherein said expensible tubular supporting element is designed and constructed for dilating a constricted blood vessel in said body vasculature.

136. The method of claim 134, wherein each of said at least one coat is independently a tubular structure.

137. The method of claim 135, wherein said at least one pharmaceutical agent serves for treating at least one disorder in said blood vessel.

138. The method of claim 137, wherein said at least one disorder comprises an injury inflicted on tissues of said blood vessel upon implantation of the stent assembly therein.

139. The method of claim 137, wherein said at least one disorder is selected from the group consisting of restenosis and in-stent stenosis.

140. The method of claim 137, wherein said at least one disorder is hyper cell proliferation.

141. The method of claim 134, wherein said at least one coat and said at least one pharmaceutical agent are configured and designed so as to provide a predetermined sustained release rate for effecting said delivery.

142. The method of claim 134, wherein said at least one coat and said at least one pharmaceutical agent are configured and designed so as to provide a predetermined duration of said delivery.

143. The method of claim 134, wherein said delivery is by diffusion.

144. The method of claim 143, wherein said delivery is initiated by a radial stretch of said at least one coat, said radial stretch is caused by an expansion of said expensible tubular supporting element.

145. The method of claim 134, wherein said expansible tubular supporting element comprises a deformable mesh of metal wires.

146. The method of claim 134, wherein said expensible tubular supporting element comprises a deformable mesh of stainless steel wires.

147. The method of claim 134, wherein said at least one coat comprises an inner coat and an outer coat.

148. The method of claim 147, wherein said inner coat comprises a layer lining an inner surface of said expansible tubular supporting element.

149. The method of claim 147, wherein said outer coat comprises a layer covering an outer surface of said expensible tubular supporting element.

150. The method of claim 134, wherein said electrospun polymer fibers are made of a biocompatible polymer.

151. The method of claim 134, wherein at least a portion of said electrospun polymer fibers is made of a biodegradable polymer.

152. The method of claim 134, wherein at least a portion of said electrospun polymer fibers is made of a biostable polymer.

153. The method of claim 134, wherein at least a portion of said electrospun polymer fibers is made of a combination of a biodegradable polymer and a biostable polymer.

154. The method of claim 134, wherein said electrospun polymer fibers are manufactured from a liquefied polymer.

155. The method of claim 154, wherein said at least one pharmaceutical agent is dissolved in said liquefied polymer.

156. The method of claim 154, wherein said at least one pharmaceutical agent is suspended in said liquefied polymer.

157. The method of claim 134, wherein said at least one pharmaceutical agent is constituted by compact objects distributed between said electrospun polymer fibers of said at least one coat.

158. The method of claim 157, wherein said compact objects are capsules.

159. The method of claim 134, wherein said at least one pharmaceutical agent is constituted by particles embedded in said electrospun polymer fibers.

160. The method of claim 134, wherein said at least one coat includes an adhesion layer.

161. The method of claim 160, wherein said adhesion layer is impervious adhesion layer.

162. The method of claim 160, wherein said adhesion layer is formed from electrospun polymer fibers.

163. The method of claim 134, wherein said electrospun polymer fibers are selected from the group consisting of polyethylene-terephtalat fibers and polyurethane fibers.

164. The method of claim 134, wherein said at least one pharmaceutical agent comprises heparin or heparin derivative.

165. The method of claim 134, wherein said at least one pharmaceutical agent comprises a radioactive compound.

166. The method of claim 134, wherein said at least one pharmaceutical agent comprises silver sulfadiazine.

167. The method of claim 134, wherein said at least one pharmaceutical agent comprises an antiproliferative drug.

168. The method of claim 134, wherein said at least one pharmaceutical agent comprises an anticoagulant drug.

169. The method of claim 145, wherein said at least one coat exposes gaps between said metal wires and exclusively covers said metal wires.

170. The method of claim 145, wherein said at least one coat substantially covers both gaps between said metal wires and said metal wires.

171. A method of coating a medical implant, implantable in a body, and loading the medical implant with a pharmaceutical agent, the method comprising:
(a) electrospinning a first liquefied polymer onto the medical implant, thereby coating the medical implant with a first coat having a predetermined porosity; and
(b) incorporating at least one pharmaceutical agent into said first coat;
thereby providing a coated medical implant loaded with the at least one pharmaceutical agent.

172. The method of claim 171, wherein the medical implant is selected from the group consisting of a graft, a patch and a valve.

173. The method of claim 171, wherein said at least one pharmaceutical agent is mixed with a liquefied polymer prior to said step of electrospinning, hence said step of incorporating said at least one pharmaceutical agent into said first coat is concomitant with said electrospinning.

174. The method of claim 173, wherein said at least one pharmaceutical agent is dissolved in said in said first liquefied polymer.

175. The method of claim 173, wherein said at least one pharmaceutical agent is suspended in said first liquefied polymer.

176. The method of claim 173, wherein said at least one pharmaceutical agent is constituted by particles embedded in polymer fibers produced during said step of electrospinning.

177. The method of claim 171, wherein said step of incorporating at least one pharmaceutical agent into said first coat comprises constituting said at least one pharmaceutical agent into compact objects, and distributing said compact objects between polymer fibers produced during said step of electrospinning.

178. The method of claim 177, wherein said compact objects are capsules.

179. The method of claim 177, wherein said compact objects are in a powder form.

180. The method of claim 177, wherein said distributing of said compact objects is by spraying.

181. The method of claim 171, wherein said coat is of a tubular structure.

182. The method of claim 171, further comprising rotating the medical implant during said step (a).

183. The method of claim 182, wherein said rotating comprises connecting the medical implant to a rotating mandrel.

184. The method of claim 183, further comprising electrospinning a second liquefied polymer onto said mandrel, prior to said step (a), hence providing an inner coat.

185. The method of claim 171, further comprising electrospinning at least one additional liquefied polymer onto said first coat, hence providing at least one additional coat.

186. The method of claim 171, further comprising providing at least one adhesion layer onto the medical implant.

187. The method of claim 184, further comprising providing at least one adhesion layer onto at least one coat.

188. The method of claim 186, wherein said adhesion layer is an impervious adhesion layer.

189. The method of claim 187, wherein said adhesion layer is an impervious adhesion layer.

190. The method of claim 186, wherein said providing at least one adhesion layer is by electrospinning.

191. The method of claim 187, wherein said providing at least one adhesion layer is by electrospinning.

192. The method of claim 183, wherein said electrospinning step comprises:
(i) charging said liquefied polymer thereby producing a charged liquefied polymer;
(ii) subjecting said charged liquefied polymer to a first electric field; and
(iii) dispensing said charged liquefied polymers within said first electric field in a direction of said mandrel.

193. The method of claim 192, wherein said mandrel is of a conductive material.

194. The method of claim 193, wherein said first electric field is defined between said mandrel and a dispensing electrode being at a first potential relative to said mandrel.

195. The method of claim 193, further comprising providing a second electric field defined by a subsidiary electrode being at a second potential relative to said mandrel, said second electric field being for modifying said first electric field.

196. The method of claim 195, wherein said subsidiary electrode serves for reducing non-uniformities in said first electric field.

197. The method of claim 195, wherein said subsidiary electrode serves for controlling fiber orientation of each of said coats generated upon the medical implant.

198. The method of claim 192, wherein said mandrel is of a dielectric material.

199. The method of claim 192, wherein the medical implant serves as a mandrel.

200. The method of claim 198, wherein said first electric field is defined between the medical implant and a dispensing electrode being at a first potential relative to the medical implant.

201. The method of claim 198, further comprising providing a second electric field defined by a subsidiary electrode being at a second potential relative to the medical implant, said second electric field being for modifying said first electric field.

202. The method of claim 201, wherein said subsidiary electrode serves for reducing non-uniformities in said first electric field.

203. The method of claim 201, wherein said subsidiary electrode serves for controlling fiber orientation of each of said coats generated upon the medical implant.

204. The method of claim 171, wherein said first liquefied polymer is a biocompatible liquefied polymer.

205. The method of claim 171, wherein said first liquefied polymer is a biodegradable liquefied polymer.

206. The method of claim 171, wherein said first liquefied polymer is a biostable liquefied polymer.

207. The method of claim 171, wherein first liquefied polymer is a combination of a biodegradable liquefied polymer and a biostable liquefied polymer.

208. The method of claim 184, wherein said second liquefied polymer is a biocompatible liquefied polymer.

209. The method of claim 184, wherein said second liquefied polymer is a biodegradable liquefied polymer.

210. The method of claim 184, wherein said second liquefied polymer is a biostable liquefied polymer.

211. The method of claim 184, wherein said second liquefied polymer is a combination of a biodegradable liquefied polymer and a biostable liquefied polymer.

212. The method of claim 185, wherein each of said at least one additional liquefied polymer is independently a biocompatible liquefied polymer.

213. The method of claim 185, wherein each of said at least one additional liquefied polymer is independently a biodegradable liquefied polymer.

214. The method of claim 185, wherein each of said at least one additional liquefied polymer is independently a biostable liquefied polymer.

215. The method of claim 185, wherein each of said at least one additional liquefied polymer is independently a combination of a biodegradable liquefied polymer and a biostable liquefied polymer.

216. The method of claim 171, wherein said at least one pharmaceutical agent is Heparin.

217. The method of claim 171, wherein said at least one pharmaceutical agent is a radioactive compound.

218. The method of claim 171, wherein said at least one pharmaceutical agent is silver sulfadiazine.

219. The method of claim 183, further comprising heating said mandrel prior to, during or subsequent to said step of electrospinning.

220. The method of claim 219, wherein said heating of said mandrel is selected from the group consisting of external heating and internal heating.

221. The method of claim 220, wherein said external heating is by at least one infrared radiator.

222. The method of claim 221, wherein said at least one infrared radiator is an infrared lamp.

223. The method of claim 220, wherein said internal heating is by a built-in heater.

224. The method of claim 223, wherein said built-in heater is an Ohmic built-in heater.

225. The method of claim 183, further comprising removing the coated medical implant from said mandrel.

226. The method of claim 225, further comprising dipping the coated medical implant in a vapor.

227. The method of claim 226, further comprising heating said vapor.

228. The method of claim 225, wherein said vapor is saturated a DMF vapor.

229. The method of claim 171, further comprising exposing the coated medical implant to a partial vacuum processing.
